(19) United States
(12) Patent Application Publication
(10) Pub. No.: US 2013/0259906 A1
(43) Pub. Date: Oct. 3, 2013

(54) PHARMACEUTICAL COMPOSITION COMPRISING ONE OR MORE FUMARIC ACID ESTERS

(71) Applicant: FORWARD PHARMA A/S, Copenhagen K (DK)

(72) Inventors: Henrik Nilsson, Vienna (AT); Roland Rupp, Bergisch-Gladbach (DE)

(73) Assignee: Forward Pharma A/S, Copenhagen K (DK)

(21) Appl. No.: 13/768,829

(22) Filed: Feb. 15, 2013

Related U.S. Application Data
(63) Continuation of application No. 13/143,465, filed on Oct. 26, 2011, now abandoned.
(60) Provisional application No. 61/143,515, filed on Jan. 9, 2009.

(30) Foreign Application Priority Data
Jan. 9, 2009 (DK) PA 2009 00033

(51) Int. Cl. A61K 31/223

(52) U.S. Cl. CPC A61K 31/223
USPC 424/400; 560/201; 514/547

(57) ABSTRACT

A pharmaceutical controlled release composition comprising one or more fumaric acid esters.

### FIELD OF THE INVENTION

The present invention relates to a pharmaceutical controlled or sustained release composition comprising one or more fumaric acid esters.

### BACKGROUND OF THE INVENTION

Psoriasis is a chronic skin disease, with a high percentage of genetic pre-disposition. The disease fluctuates between acute exacerbation and times of complete standstill. Patients suffering from psoriasis may be severely handicapped because of the external characteristics of the disease. This affects all parts of life, such as the professional career as well as the personal and private life.

The therapeutic possibilities available until to date have been limited, in particular for patients with moderate to severe psoriasis, and many of them provide only a temporary and short-term improvement, and/or are associated with severe side effects. Since psoriasis has a high recurrence rate, the majority of patients have to undergo long-term treatment.

Fumaric acid esters have been used for the treatment of moderate to severe psoriasis for more than 30 years. In 1994 a defined mixture of dimethyl fumarate and monoethyl fumarate salts was approved in Germany—Fumaderm® initial/Fumaderm®. One enteric coated tablet of Fumaderm® contains the following active ingredients: dimethylfumarate 120 mg; ethylhydrogenfumarate, calcium salt 87 mg; ethylhydrogenfumarate, magnesium salt 5 mg; ethylhydrogenfumarate, zink salt 3 mg, and the following other ingredients: croscarmellose-sodium, talc, magnesium stearate, coloring agents E 171 and E 132, methacrylic acid-methylmethacrylate-copolymer (1:1), methacrylic acid-ethylacrylate-copolymer (1:1), Macrogol 6000, simethicone, povidone, trlethyl citrate, microcrystalline cellulose, highly disperse silicon dioxide [Summary of Product Characteristics, Fumaderm®, version January 2009]. By today Fumaderm® represents about 66% of all prescriptions for systemic therapy of psoriasis in Germany. However, a high frequency of side effects, e.g. gastrointestinal side effects, causes some patient discontinuation early in treatment. It is contemplated that the gastrointestinal side effects and flushing can, at least partially, be explained by the immediate release properties of the prescription formulation, leading to high local concentrations in the intestines.

Fumaric acid esters, such as dimethyl fumarate, can be subject to degradation and hydrolysis. It is e.g. known that dimethyl fumarate is more prone to hydrolysis in an alkaline/less acidic environment, c.f. more acidic environments (Litjens et al, “In vitro pharmacokinetics of anti-psoriatic fumaric acid esters”, BMC Pharmacology 2004, 4:22). Thus, dimethyl fumarate is considered to be more prone to hydrolysis in the small intestine, c.f. the gastric ventricle. In addition to the pH effect described above, esterases are considered to contribute to hydrolysis of fumaric acid esters.

The present inventors contemplate that an improved treatment regimen may be obtained by administration of a pharmaceutical composition that is designed to deliver the active substance in a controlled manner, i.e. in a manner that is prolonged, slow and/or delayed compared with the commercially available product.

### OBJECT OF THE INVENTION

It is an object of embodiments of the invention to provide controlled or sustained release of the API to improve the tolerability c.f. the commercially available formulation. In one embodiment, the composition according to the invention is an erosion matrix system, whereby the exposure of API to hydrolysis and enzymes within the gastrointestinal tract is contemplated to be minimized, thereby mitigating degradation of the API.

It is a further object of embodiments of the invention to provide a controlled or sustained release pharmaceutical formulation comprising fumaric acid ester(s) as active substance(s) wherein the controlled release composition results in a reduction in GI (gastro-intestinal) related side-effects and/or a reduction in flushing and/or wherein a reduced variability e.g. compared to the prior art Fumaderm® formulation may be obtained, and/or an adequate relative bioavailability c.f. e.g. the prior art Fumaderm® formulation may be obtained, and/or an increased relative bioavailability c.f. e.g. the prior art Fumaderm® formulation may be obtained.

### SUMMARY OF THE INVENTION

It has been found by the present inventor(s) that compositions with in vitro release according to the invention in one embodiment can result in advantageous pharmacokinetic properties. In another embodiment, advantageous tolerability (such as less side effects) is achieved.

So, in a first aspect the present invention relates to a pharmaceutical composition comprising as an active substance one or more fumaric acid esters selected from di-(C₁-C₅)alkylesters of fumaric acid and mono-(C₁-C₅)alkylesters of fumaric acid, or a pharmaceutically acceptable salt thereof, wherein the release of the fumaric acid ester—when subjected to an in vitro dissolution test employing 0.1 N hydrochloric acid as dissolution medium during the first 2 hours of the test and then 0.05 M phosphate buffer pH 6.8 as dissolution medium—is as follows:

within the first 2 hours after start of the test from about 0% w/w to about 60% w/w of the fumaric ester contained in the formulation is released, and/or
within the first 3 hours after start of the test from about 75% w/w to about 100% w/w, such as from about 75% w/w to about 95% w/w of the total amount of the fumaric acid ester contained in the formulation is released.

In another aspect the present invention relates to method of treating psoriasis, psoriatic arthritis, neurodermatitis, inflammatory bowel disease, such as Crohn's disease and ulcerative colitis, polyarthritis, multiple sclerosis (MS), juvenile-onset diabetes mellitus, Hashimoto's thyroiditis, Grave's disease, SLE (systemic lupus erythematosus), Sjögren's syndrome, Pernicious anemia, Chronic active (lupoid) hepatitis, Rheumatoid arthritis (RA), lupus nephritis, myasthenia gravis, uveitis, refractory uveitis, vernal conjunctivitis, pemphigus vulgaris, scleroderma, optic neuritis, pain such as radicular pain, pain associated with radiculopathy, neuropathic pain or sciatica/sciatic pain, organ transplantation (prevention of rejection), sarcoidosis, necrobiosis lipoidica or granuloma annulare, which method comprises administering orally to a patient in need thereof, an effective dosage of a pharmaceutical composition according to the invention.

In another aspect the present invention relates to a use of a pharmaceutical composition according to the invention for the preparation of a medicament for the treatment of psoriasis, psoriatic arthritis, neurodermatitis, inflammatory bowel disease, such as Crohn's disease and ulcerative colitis, polyarthritis, multiple sclerosis (MS), juvenile-onset diabetes mellitus, Hashimoto's thyroiditis, Grave's disease, SLE (systemic lupus erythematosus), Sjögren's syndrome, Pernicious anemia, Chronic active (lupoid) hepatitis, Rheumatoid arthritis (RA), lupus nephritis, myasthenia gravis, uveitis, refractory uveitis, vernal conjunctivitis, pemphigus vulgaris, scleroderma, optic neuritis, pain such as radicular pain, pain associated with radiculopathy, neuropathic pain or sciatica/sciatic pain, organ transplantation (prevention of rejection), sarcoidosis, necrobiosis lipoidica or granuloma annulare.

### LEGENDS TO THE FIGURES

FIG. 1 shows the in vitro dissolution profile at 37° C. using a paddle dissolution apparatus at 100 rpm employing 0.1 N hydrochloric acid as dissolution medium during the first 2 hours of the test and then followed by 0.05 M phosphate buffer pH 6.8 as dissolution medium for the remaining test period of an enteric coated tablet (Example 1) according to the invention, and

FIG. 2 shows the in vitro dissolution profile at 37° C. using a paddle dissolution apparatus at 100 rpm employing 0.1 N hydrochloric acid as dissolution medium during the first 2 hours of the test and then followed by 0.05 M phosphate buffer pH 6.8 as dissolution medium for the remaining test period of a film coated tablet (Example 2) according to the invention.

### DETAILED DISCLOSURE OF THE INVENTION

In the present context the term “API”, which is an abbreviation for “active pharmaceutical ingredient” and the term “active substance” are used interchangeably and refer to the fumaric acid ester that is to be released from the pharmaceutical formulation according to the invention.

In the present context, the term “controlled release” refers to the release from a formulation that is designed to release the fumaric acid ester in a prolonged, slow, retarded and/or delayed manner compared to the release of the commercially available product Fumaderm®, when tested under comparable conditions (e.g. for in vivo studies: dose equivalents, with or without standardized meal etc., or for in vitro studies: dose equivalents, dissolution test apparatus and working conditions including e.g. composition, volume and temperature of dissolution medium employed, rotation speed etc.).

In the present context the term “variability” refers to the variability of PK parameters (e.g. Cmax and AUC) after administration of a pharmaceutical formulation or a reference formulation. The variability can be expressed as the coefficient of variation (CV) for a PK parameter, i.e. the ratio of the standard deviation to the mean.

It has been found that formulations according to the invention exhibit a relatively good in vitro/In vivo correlation. In an aspect the in vitro/in vivo correlation is determined by comparing the time to 80% of the fumaric acid ester being released from the formulations in an in vitro dissolution test to the Cmaxbeing measured in vivo after administration of the formulations.

The release in vivo may be tested by measuring the plasma concentration at predetermined time periods and thereby obtaining a plasma concentration versus time profile for the fumaric acid ester in question or, if relevant, a metabolite thereof. (E.g. in the case of dimethylfumarate, the active substance is envisaged to be methylhydrogenfumarate, i.e. the monomethyl ester of fumaric acid). Furthermore, it is contemplated that metabolism already takes place within the gastro-intestinal tract or during passage of the gastro-intestinal mucosa, or upon first passage through the hepatic circulation. Accordingly, when dimethylfumarate is administered, the relevant component to search for in the plasma may be the monomethyl ester and not the dimethylester of fumaric acid.

Other tests may also be used to determine or to give a measure of the release of the active substance in vivo. Thus, animals (e.g. minipigs, dogs etc.) may be used as a model. The animals receive the compositions under investigation and after specified periods of time, blood samples are collected and the content of the active ingredient (or metabolite thereof, if relevant) is determined in plasma or specific organs or extracted from the intestinal contents.

Another test involves the use of a specific segment of an animal intestine. The segment is placed in a suitable apparatus containing two compartments (a donor and a receiver) separated by the segment, and the composition under investigation is placed in a suitable medium in one compartment (the donor compartment). The composition will release the active substance that subsequently is transported across the intestinal segment. Accordingly, at suitable time intervals, the concentration of the active substance (or, if relevant, the metabolite) is measured in the receiver compartment.

A person skilled in the art will be able to adapt the above-mentioned method to the specific composition.

With respect to in vitro methods, well-established methods are available, especially methods described by official monographs like e.g. United States Pharmacopeia (USP) or the European Pharmacopoeia. A person skilled in the art will know which method to choose and how to select the specific conditions to carry out the in vitro test. For instance, the USP prescribes in vitro tests be carried out at 37+/−1.0 such as 37+/−0.5 degrees Celsius/Centigrade. In one aspect, a suitable dissolution test is one, wherein the dissolution profile is determined as described in the United States Pharmacopoeia at 37° C. using a paddle dissolution apparatus at 100 rpm employing 0.1 N hydrochloric acid as dissolution medium during the first 2 hours of the test and then followed by 0.05 M phosphate buffer pH 6.8 as dissolution medium for the remaining test period. A person skilled in the art will know how to adjust the conditions applied, e.g. temperature, pH, paddle speed, duration etc. In a further aspect, the in vitro dissolution testing is carried out as follows: A USP apparatus II (paddles) with 1 litre vessels is used. Bath temperature is set to 37° C.±0.5° C. and paddle speed to 100 rpm. One tablet is placed in one vessel containing 750 ml 0.1N HCl (pH 1.2) over 2 h. After that the pH is changed to 6.8 by adding 220 ml 0.2 M sodium phosphate buffer. 1.5 ml samples are taken at each sampling time point and analyzed by HPLC for DMF. The HPLC parameters are set as follows: Column: Phenomenex Luna C18, 50×4.6 mm, 3 μm; column oven temperature 30° C.: mobile phase: Methanol: 20 mM phosphate buffer pH 3.0 (35:65 V/V), inject volume: 5 μl, Flow rate: 0.8 ml/min, Detector wavelength: 210 nm, run time 5 min, DMF retention time 3.5 min.

In the present context the term “rate-controlling agent” refers to an agent that is able to delay and/or prolong the in vivo release of the active substance.

As mentioned above, the in vivo release of the active substance is prolonged, slow and/or delayed compared with the commercially available Fumaderm® composition. In the present context, the term “prolonged” is intended to indicate that the active substance is released during a longer time period than Fumaderm® such as at least during a time period that is at least 1.2 times, such as, e.g., at least 1.5 times, at least 2 times, at least 3 times, at least 4 times or at least 5 times greater than that of Fumaderm®. Thus, if e.g. 100% of dimethylfumarate is released from Fumaderm® tablets 3 hours after the start of a suitable test, then 100% of dimethylfumarate in a composition according to the invention is released at least 3.6 hours after the start of a suitable test.

In the present context the term “delayed” is intended to indicate that the release of the active substance starts at a later point in time compared with that of Fumaderm® (such as at 30 min or more later such as, e.g., 45 min or more later, 1 hour or more later or 1.5 hours or more later.

In the present context the term “monolithic” refers to consisting of or constituting a single unit.

The formulation according to the invention is contemplated to provide improved tolerability, such as fewer and/or less severe gastrointestinal (GI) side-effects, such as fewer and/or less severe redness episodes, such as fewer and/or less severe flushing episodes.

As used in the present invention, a gastrointestinal (GI) side effect may include, but is not limited to diarrhea, stomach ache, stomach pain, abdominal pain, abdominal cramps, nausea, flatulence, tenesmus, meteorism, an increased frequency of stools, a feeling of fullness and upper abdominal cramps. In the present context, a reduction of GI related side effects is intended to denote a decrease in severity and/or incidence among a given treated patient population, comparing the GI side effects observed after administration of the composition according to the invention to the GI side effects observed after administration of Fumaderm®. A reduction in GI related side effects according to this definition could thus be construed as a substantial reduction in incidence of any of the GI side effect listed above, such as at least a 10% reduction in incidence or more preferably at least 20% reduction in incidence or even more preferable a more than 30% reduction in incidence. A reduction in GI related side effect can also be expressed as a substantial reduction in severity in any of the GI side effects listed above, such as a reduction in severity and/or frequency of diarrhea, stomach ache, stomach pain, abdominal pain, abdominal cramps, nausea, flatulence, tenesmus, meteorism, increased frequency of stools, a feeling of fullness or upper abdominal cramps. The reduction of GI related side effects, as described above, can be monitored in a clinical trial setting, either comparing the administration of the composition according to the invention head on with Fumaderm® or with placebo. In case of a placebo controlled trial, the incidence of GI related side effects in the patients receiving the composition according to the invention compared to the placebo group, can be compared to historical trials comparing Fumaderm® to placebo (see e.g. Altmeyer et al, J. Am. Acad. Dermatol. 1994; full reference: Altmeyer P J et al, Antipsoriatic effect of fumaric acid derivatives. Results of a multicenter double-blind study in 100 patients. 3. Am. Acad. Dermatol. 1994; 30:977-81).

In a further aspect, the formulation according to the invention—upon oral administration and in comparison to that obtained after oral administration of Fumaderm® tablets in an equivalent dosage—reduce (GI) side-effects (frequency and/or severity).

In one embodiment, such a clinical trial can be carried out as described below under “Clinical trial in patients”. In another embodiment, such a clinical trial can be carried out as described below under “Clinical trial in healthy volunteers”.

### Clinical Trial in Patients:

Typically, patients suffering from psoriasis are included in such a study, and typically more than 10% of the body surface area will be affected by psoriasis (severe psoriasis). However, patients in whom between 2 and 10 percent of the body surface area is affected can also be included (moderate psoriasis). Patients can also be selected based on the psoriasis area severity index (PASI) score. Typically, patients within a certain range of PASI scores are included, such as between 10 and 40, or such as between 12 and 30, or such as between 15 and 25. In another embodiment, patients with a certain minimum PASI score are included, such as a PAST score of at least 8, such as at least 10, such as at least 12, such as at least 15. Patients with any type of psoriasis may be included (chronic plaque type, exanthematic guttate type, pustular type, psoriatic erythroderma or palmoplantar type), but in some cases only patients with the chronic plaque type are included. About 15 to 20 patients in each treatment group (composition according to the invention, Fumaderm® or placebo) are sufficient in most cases, but more preferably about 30 to 50 patients are included in each arm of the study. Total study duration can be as short as one day to one week, but more preferably the study will run for 8 weeks to 12 weeks or up to 16 weeks or longer. The side effects can e.g. be assessed as the total number of times a certain side effect was reported in each group (irrespective of how many patients have experienced the side effect), or the side effects can be assessed as the number of patients that have experienced a certain side effect a certain number of times, such as at least once or at least twice or at least three times during the duration of the study. Furthermore, the severity of a side effect can be monitored, or a certain severity of a side effect can be required for it to qualify as a side effect in the study. A convenient way of assessing the severity of a side effect is via a visual analogue (VAS) scale.

### Clinical Trial in Healthy Volunteers:

This study will typically be a single center study, following an open-label, randomized, crossover design to investigate the plasma concentrations, pharmacokinetics, safety and tolerability of pharmaceutical formulations according to the invention (in this case three different ones), possibly c.f. the marketed formulation Fumaderm® as reference. The tablets will be administered as a single oral dose of 240 mg (2 tablets containing 120 mg each) in each treatment period according to randomization to 20 healthy, male Caucasian subjects. The study is divided into four treatment periods (Treatment Period 1, 2, 3 and 4), which will be separated by a wash-out phase of at least 7 days.

Subjects will be screened for eligibility at least 21 to 2 days before first administration including: check of inclusion/exclusion criteria; demographic data (including age, body height, body weight, body mass index (BMI), and ethnic origin); physical examination; complete medical history; 12-lead electrocardiogram (ECG); vital signs (blood pressure (BP), pulse rate (PR), and body temperature (BT)); clinical laboratory parameters (hematology, serum biochemistry, and urinalysis); documentation of concomitant illness and medication. At each of the four treatment periods, subjects will come to the Study Site in the evening of Day −1 at approximately 06:00 p.m. (or earlier, if additional testing is required on Day −1) and will remain there until the 24-hour blood sample for PK analysis is drawn and all safety measurements are performed (=morning of Day 2).

The subjects will fast overnight. A single oral dose (of two tablets) of one of the formulations according to the invention, or two tablets of the reference medication Fumaderm® each containing 120 mg dimethyl fumarate (total dose 240 mg dimethyl fumarate) will be administered on Day 1 (according to randomization). Administration will be done to subjects who are in fasting condition together with 240 ml tap water. Between each administration, a wash-out interval of at least 7 days will be maintained.

### The Following Assessments/Measurements Will be Performed:

Blood sampling will be performed for the determination of plasma concentrations and PK-parameters prior to, and at pre-scheduled times post dosing.
Urine will be collected prior to and at pre-scheduled times post dosing.
A follow-up examination will be performed at least 7 days after the last administration (Treatment Period 4), including: physical examination; vital signs (BP, PR, and BT); body weight; 12-lead ECG; clinical laboratory parameters (haematology, serum biochemistry, and urinalysis); documentation of concomitant medication and adverse events.

In a further aspect, the composition according to the invention—upon oral administration and in comparison to that obtained after oral administration of Fumaderm® tablets in an equivalent dosage—reduce flushing (frequency and/or severity).

In the present context the term “flushing” describes episodic attacks of redness of the skin together with a sensation of warmth or burning of the face, neck, and less frequently the upper trunk and abdomen. It is the transient nature of the attacks that distinguishes flushing from the persistent erythema of photosensitivity or acute contact reactions. Repeated flushing over a prolonged period of time can lead to telangiectasla and occasionally to classical rosacea of the face (Greaves M W. Flushing and flushing syndromes, rosacea and perioral dermatitis. In: Champion R H, et al, eds. Rook/Wilkinson/Ebling textbook of dermatology, 6th ed., vol. 3. Oxford, UK: Blackwell Scientific, 1998: 2099-2104).

In the present context, a reduction of flushing is intended to denote a decrease in severity and/or incidence/frequency among a given treated patient population of flushing observed after administration of the composition according to the invention compared with flushing observed after administration of Fumaderm® and can be measured e.g as described by O'toole et al. Cancer 2000, 88(4): p. 770-776. A reduction in flushing according to this definition could thus be construed as a reduction in incidence and/or severity of flushing. In one aspect of the invention, the incidence of flushing is reduced by at least about a quarter, in another aspect of the invention the incidence is reduced by at least about a third, in another aspect of the invention the incidence is reduced by at least about half, and in a further aspect of the invention, the flushing incidence is reduced by about two thirds or more. Likewise, the severity is in one aspect of the invention reduced by at least about a quarter, in another aspect of the invention by at least about a third, in another aspect of the invention by at least half, and in a further aspect of the invention by at least about two thirds. A one hundred percent reduction in flushing incidence and severity is mast preferable, but is not required. The reduction of flushing, as described above, can be monitored in a clinical trial setting, e.g. comparing the administration of the compound according to the invention with e.g. administration of Fumaderm®. In case of a Fumaderm® controlled trial, the incidence and severity, defined as mild, moderate or severe, of flushing in the patients receiving the compound according to the invention compared to the Fumaderm® group, can be compared.

In one aspect, the severity of flushing is determined as the body surface area involved.

In one embodiment, such a clinical trial can be carried out as described above under “Clinical trial in patients”. In another embodiment, such a clinical trial can be carried out as described above under “Clinical trial in healthy volunteers”.

In a further aspect, the composition according to the invention—upon oral administration and in comparison to that obtained after oral administration of Fumaderm® tablets in an equivalent dosage—reduce redness (frequency and/or severity).

In the present context the term “redness” describes episodic attacks of redness of the skin. In one aspect, the redness occurs in the face, neck, and less frequently the upper trunk and abdomen.

In the present context, a reduction of redness is intended to denote a decrease in severity and/or incidence/frequency among a given treated patient population of redness observed after administration of the composition according to the invention compared with redness observed after administration of Fumaderm® and can e.g. be assessed by a clinician or nurse. A reduction in redness according to this definition could thus be construed as a reduction in incidence and/or severity of redness. In one aspect of the invention, the incidence of redness is reduced by at least about a quarter, in another aspect of the invention the incidence is reduced by at least about a third, in another aspect of the invention the incidence is reduced by at least about half, and in a further aspect of the invention, the redness incidence is reduced by about two thirds or more. Likewise, the severity is in one aspect of the invention reduced by at least about a quarter, in another aspect of the invention by at least about a third, in another aspect of the invention by at least half, and in a further aspect of the invention by at least about two thirds. A one hundred percent reduction in redness incidence and severity is most preferable, but is not required. The reduction of redness, as described above, can be monitored in a clinical trial setting, e.g. comparing the administration of the compound according to the invention with e.g. administration of Fumaderm®. In case of a Fumaderm® controlled trial, the incidence and severity, defined as mild, moderate or severe, of redness in the patients receiving the compound according to the invention compared to the Fumaderm® group, can be compared.

In one aspect, the severity of redness is determined as the body surface area involved.

In one embodiment, such a clinical trial can be carried out as described above under “Clinical trial in patients”. In another embodiment, such a clinical trial can be carried out as described above under “Clinical trial in healthy volunteers”.

In the present context the term “erosion matrix” refers to a matrix wherein the release of the API does not depend upon intrinsic diffusion processes but rather is the result of the rate of the matrix erosion. By stripping off the erosion matrix layers in a well controlled manner predetermined amounts of the API will be obtained with the release of API being dependent on the rate of swelling and dissolution or erosion of the matrix and on the rate of dissolution, solubility and rate of diffusion of the API.

An embodiment of the invention is a composition, wherein the release of the fumaric acid ester, when subjected to an in vitro dissolution test employing 0.1 N hydrochloric acid as dissolution medium during the first 2 hours of the test and then 0.05 M phosphate buffer pH 6.8 as dissolution medium—is as follows:

within the first 2 hours after start of the test from about 0% w/w to about 60% w/w, such as about 10% w/w to about 60% w/w, such as about 20% w/w to about 50% w/w, such as about 30% w/w to about 50%, such as about 40% w/w to about 50% w/w of the fumaric ester contained in the formulation is released, and/or
within the first 3 hours after start of the test from about 75% w/w to about 100% w/w, such as from about 75% w/w to about 95% w/w, such as from about 80% w/w to about 100% w/w, such as from about 80% w/w to about 95% w/w, such as from about 85% w/w to about 100% w/w, such as from about 85% w/w to about 95% w/w, such as from about 90% w/w to about 100% w/w, such as from about 90% w/w to about 95 w/w of the fumaric ester contained in the formulation is released.

An embodiment of the invention is a composition, wherein the release of the fumaric acid ester, when subjected to an in vitro dissolution test employing 0.1 N hydrochloric acid as dissolution medium during the first 2 hours of the test and then 0.05 M phosphate buffer pH 6.8 as dissolution medium—is as follows: within the first 4 hours after start of the test about 92% to about 100%, such as about 94% w/w to about 98% w/w, such as about 95% w/w of the total amount of the fumaric acid ester contained in the formulation is released.

An embodiment of the invention is a composition, wherein the release of the fumaric acid ester, when subjected to an in vitro dissolution test employing 0.1 N hydrochloric acid as dissolution medium during the first 2 hours of the test and then 0.05 M phosphate buffer pH 6.8 as dissolution medium—is as follows:

within the first 5 hours after start of the test about 94% to about 100%, such as about 94% w/w to about 99% w/w, such as about 95% to 98% of the total amount of the fumaric acid ester contained in the formulation is released.

An embodiment of the invention is a composition, wherein the release of the fumaric acid ester, when subjected to an in vitro dissolution test employing 0.1 N hydrochloric acid as dissolution medium during the first 2 hours of the test and then 0.05 M phosphate buffer pH 6.8 as dissolution medium—is as follows:

within the first 6 hours after start of the test about 95% to about 100%, such as about 96 w/w to about 99% w/w, such as about 97% to 98% of the total amount of the fumaric acid ester contained in the formulation is released.

An embodiment of the invention is a composition, wherein the release of the fumaric acid ester—when subjected to an in vitro dissolution test employing 0.1 N hydrochloric acid as dissolution medium during the first 2 hours of the test and then 0.05 M phosphate buffer pH 6.8 as dissolution medium—is as follows:

within the first 2 hours after start of the test from about 40% w/w to about 50% w/w of the fumaric ester contained in the formulation is released, and
within the first 3 hours after start of the test from about 85% w/w to about 95% w/w of the total amount of the fumaric acid ester contained in the formulation is released; and
within the first 4 hours after start of the test from about 92% w/w to about 100% w/w of the total amount of the fumaric acid ester contained in the formulation is released.

An embodiment of the invention is a composition, wherein the release of the fumaric acid ester—when subjected to an in vitro dissolution test employing 0.1 N hydrochloric acid as dissolution medium during the first 2 hours of the test and then 0.05 M phosphate buffer pH 6.8 as dissolution medium—is as follows:

within the first 2 hours after start of the test from about 40% w/w to about 50% w/w of the fumaric ester contained in the formulation is released, and

within the first 3 hours after start of the test from about 85% w/w to about 100% w/w of the total amount of the fumaric acid ester contained in the formulation is released; and

within the first 4 hours after start of the test from about 92% w/w to about 100% w/w of the total amount of the fumaric acid ester contained in the formulation is released.

In an embodiment of the invention the release has zero order, first order or square-root (Higuchi's) kinetics release profile.

In a further embodiment the in vitro release has a combination of zero order, first order and square-root (Higuchi's) kinetics in vitro release profiles, e.g. a combination of zero and first order in vitro release profiles.

Different kinetic models, such as zero-order (1), first-order (2), square-root (Higuchi's equation) (3) can be applied to the interpretation of the drug release kinetic.

Mₜ=M₀+k₀*t 1:

InMₜ=InM+k₁*t 2:

Mₜ=M₀+k_{H}*t^{1/2} 3:

In these equations, Mₜ is the cumulative amount of drug released at any specified time point and M₀ is the dose of active substance incorporated in the pharmaceutical composition. k₀, k₁ and k_{H} are rate constants for zero-order, first-order and Higuchi's equation, respectively.

One aspect of the invention relates to a zero-order dissolution release profile. Another aspect relates to a first-order dissolution release profile. A further aspect relates to a square-root (Higuchi's equation) dissolution release profile.

In one aspect, a pharmaceutical composition comprising one or more fumaric acid esters as well as one or more rate-controlling agents allowing controlled release of said fumaric acid esters, is provided.

In the present context, the term “relative bioavailability” refers to a comparison of the amount of drug absorbed (expressed as area under the curve (AUC)) after administration of two different formulations or reference product. In the present context, the amount of drug absorbed, expressed as AUC, can be detected in the form of the actual drug administered, or as a metabolite thereof. The relative bioavailabilty can be expressed as a percentage of a reference AUC, i.e. AUC %

In one embodiment, the relative bioavailability of the formulation of the invention compared to Fumaderm® is at least about 75%, such as at least about 80%, such as at least about 85%, such as at least about 90%, such as at least about 95%, such as about 100%.

In one embodiment the relative bioavailability of the formulation of the invention compared to Fumaderm® is at least about 100%, such as at least about 110%, such as at least about 120%, such as at least about 125%, such as at least about 130%.

In one embodiment the relative bioavailability of the formulation of the invention compared to Fumaderm® is at the most about 130%, such as at the most about 125%, such as at the most about 120%, such as at the most about 110%, such as the most about 100%

In an aspect of the invention, the rate-controlling agent is a water-soluble polymer.

As used herein, the term “water-soluble polymer” means a conventional polymer for pharmaceutical use, having a solubility of more than 10 mg/ml in water. Suitable water-soluble polymers includes, but are not limited too, for example, hydroxypropylmethyl cellulose, hydroxypropyl cellulose, methyl cellulose and carboxymethyl cellulose. In one aspect, the water-soluble polymer is hydroxypropyl cellulose.

As used herein, the term “water-insoluble polymer” means a conventional polymer for pharmaceutical use, having a solubility of not more than 10 mg/ml in water.

In a further aspect of the invention, the erosion matrix contains essentially no water-insoluble polymer. In yet a further aspect, the erosion matrix contains no water-insoluble polymer.

In the present context the term “essentially no” refers to a level of less than about 1%, such as less than about 0.5%, such as less than about 0.3%, such as about 0.0%.

In an aspect of the invention, the rate-controlling agent is a water-soluble polymer and the erosion matrix contains essentially no water-Insoluble polymer.

In an aspect of the invention, the rate-controlling agent is a water-soluble polymer and the erosion matrix contains no water-insoluble polymer.

In an embodiment of the invention, the rate-controlling agent is a cellulose polymer or a cellulose derivative or a mixture thereof. As non-limiting examples of a cellulose polymer or a cellulose derivative or a mixture thereof may be mentioned hydroxypropyl cellulose, hydroxypropyl methyl cellulose (HPMC), methyl cellulose, carboxymethyl cellulose and mixtures thereof.

In an embodiment of the invention, the rate-controlling agent is hydroxypropyl cellulose. Many different grades of hydroxypropyl cellulose exist depending on e.g. the molecular weight thereof, the degree of etherification, viscosity etc. Non-limiting exemplary embodiments of commercially available hydroxypropyl celluloses are obtainable from e.g. Aqualon under the trade names Klucel® HPC-L, HPC-SL, HPC-SSL, HPC-M, HPC-H etc. In an embodiment of the invention, the rate-controlling agent is hydroxypropyl cellulose having a viscosity (mPa·s) of 3.0-5.9 as measured in an aqueous solution containing 2% by weight of dry HPC at 20° C. In an embodiment of the invention, the rate-controlling agent is HPC-SL.

In another embodiment of the invention, the rate-controlling agent is an acrylic acid polymer or copolymer or a methacrylic acid polymer or copolymer or a mixture thereof or in a mixture with one or more cellulose polymers or cellulose derivatives as mentioned above. Examples of acrylic acid polymers and copolymers and methacrylic acid polymers and copolymers include, but are not limited to, ammonio methacrylate copolymer type A, ammonio methacrylate copolymer B, methacrylic acid copolymer A, methacrylic acid copolymer B, polyvinyl acetate polymer and methacrylic acetate polymer.

In an embodiment of the invention the rate-controlling agent is present in an amount of 3-35% by weight, such as about 4-15% by weight, such as about 4-10% by weight, such as about 4-6% by weight.

In another embodiment of the invention the rate-controlling agent is present in an amount of 15-40% by weight, such as about 15-25% by weight.

In another embodiment of the invention the rate-controlling agent is present in an amount of about 25-40% by weight, such as about 35-40% by weight.

The amount of rate-controlling agent varies in accordance with the specific rate-controlling agent used, the release profile aimed at, the level and nature of any excipients and additives present in the core tablet, etc.

In an embodiment of the invention the formulation further comprises a binder. In an embodiment thereof, said binder is lactose. Lactose is commercially available in a number of different grades depending i.a. on the manufacturing method used resulting in a range of particle sizes, particle size distributions etc. Examples of lactose include, but are not limited to anhydrous lactose, lactose made from alpha-lactose-monohydrate, agglomerated lactose, granulated lactose, crystalline lactose, crystalline, sieved lactose, sieved lactose (e.g. PrismaLac®, such as PrismaLac® 40), crystalline, abrasive lactose (e.g. GranuLac®, such as GranuLac® 70, GranuLac® 140, GranuLac® 200, GranuLac® 230 and GranuLac® 400), improved lactose, agglomerated lactose (e.g. Tablettose®, such as Tablettose® 70, Tablettose® 80 and Tablettose® 100), improved lactose, spraydried lactose (FlowLac®, such as FlowLac® 90 and FlowLac® 100). have similar benefits to the ones listed above for film coating. However, in addition, the active pharmaceutical ingredient may not be released in the acidic environment of the gastric ventricle, potentially protecting the gastric mucosa from irritation, if the API has an irritant potential for the gastric mucosa.

The active substance in a composition of the invention is any fumaric acid ester.

In one embodiment of the invention the fumaric acid ester is preferably selected from the group consisting of dimethylfumarate, diethylfumarate, dipropylfumarate, dibutylfumarate, dipentylfumarate, methyl-ethylfumarate, methyl-propylfumarate, methyl-butylfumarate, methyl-pentylfumarate, monomethylfumarate, monoethylfumarate, monopropylfumarate, monobutylfumarate and monopentylfumarate, including pharmaceutically acceptable salts thereof.

Pharmaceutically acceptable salts thereof comprise metal salts, such as a salt selected from alkali metal salts and alkaline earth metal salts including sodium, potassium, calcium, magnesium, strontium or zinc salts, amino acid salts etc.

In another embodiment of the invention the fumaric acid ester is present in the form of a monosaccharide ester thereof.

In a specific embodiment of the invention, the fumaric acid ester is a mono-(C₁-C₅)alkylester of fumaric acid that is present in the form of a pharmaceutically acceptable salt. Suitable salts are e.g. metal salts such as a salt selected from alkali metal salts and alkaline earth metal salts including sodium, potassium, calcium, magnesium, strontium or zinc salt.

The term (C₁-C₅)alkyl refers to a branched or un-branched alkyl group having from one to five carbon atoms inclusive, such as methyl, ethyl, 1-propyl, 2-propyl, 1-butyl, 2-butyl, 2-methyl-2-propyl, 2-methyl-1-propyl and pentyl.

In another embodiment, the composition according to the invention comprises dimethylfumarate as the active substance.

In a further embodiment, the composition according to the invention comprises monomethylfumarate as the active substance optionally in the form of a pharmaceutically acceptable salt like e.g. its sodium, potassium, calcium, magnesium, strontium and/or zinc salt.

In another embodiment, the composition according to the invention consists essentially of dimethylfumarate as the active substance.

In another embodiment, the composition according to the invention consists of dimethylfumarate as the active substance.

In a further embodiment, the composition according to the invention consists essentially of monomethylfumarate as the active substance optionally in the form of a pharmaceutically acceptable salt like e.g. its sodium, potassium, calcium, magnesium, strontium, zinc and/or amino acid salt.

In a further embodiment, the composition according to the invention consists of monomethylfumarate as the active substance optionally in the form of a pharmaceutically acceptable salt like e.g. its sodium, potassium, calcium, magnesium, strontium, zinc and/or amino acid salt.

In a further embodiment, the composition according to the invention comprises dimethylfumarate and monomethylfumarate (optionally in the form of a pharmaceutically acceptable salt like e.g. its sodium, potassium, calcium, magnesium, strontium, zinc and/or amino acid salt) as the active substances, in a weight ratio between about 1:10 and about 10:1.

In a further embodiment, the composition according to the invention consists essentially of dimethylfumarate and monomethylfumarate (optionally in the form of a pharmaceutically acceptable salt like e.g. its sodium, potassium, calcium, magnesium, strontium, zinc and/or amino acid salt) as the active substances, in a weight ratio between about 1:10 and about 10:1.

In a further embodiment, the composition according to the invention consists of dimethylfumarate and monomethylfumarate (optionally in the form of a pharmaceutically acceptable salt like e.g. its sodium, potassium, calcium, magnesium, strontium, zinc and/or amino acid salt) as the active substances, in a weight ratio between about 1:10 and about 10:1.

In an embodiment the formulation according to the invention is for administration once, twice or three times daily.

In an embodiment the formulation is for administration once daily.

In an embodiment the formulation is for administration twice daily.

The daily dosage of the controlled release pharmaceutical composition according to the invention that is administered to treat a patient depends on a number of factors among which are included, without limitation, weight and age and the underlying causes of the condition or disease to be treated, and is within the skill of a physician to determine.

In one aspect of the invention the daily dosage can be e.g. from 200 to 400 mg active substance given in one to three doses, in another aspect from 300 to 500 mg active substance given in one to three doses, in another aspect 400 to 600 mg active substance given in one to three doses, in another aspect 500 to 700 mg active substance given in one to three doses, in another aspect 600 to 800 mg active substance given in one to three doses, in another aspect 700 to 900 mg active substance given in one to three doses, in another aspect 800 to 1000 mg active substance given in one to three doses, in another aspect 900 to 1100 mg active substance given in one to three doses, in another aspect 1000 to 1200 mg active substance given in one to three doses, in another aspect 1100 to 1300 mg active substance given in one to three doses, in another aspect 1200 to 1400 mg active substance given in one to three doses and in yet another aspect 1300 to 2000 mg active substance given in one to three doses.

In one embodiment the composition according to the invention may be prepared in the form of erosion matrix tablets. Erosion matrix tablets may be obtained by granulation, followed by tabletting and optionally film and/or enteric coating of the core tablets obtained. The core can for example be made by conventional wet granulation or continuous granulation such as extrusion followed by compaction of the granules into tablets. The core may then be coated using an appropriate technology, preferably by air suspension.

In another embodiment the composition according to the invention may be prepared as e.g. diffusion-controlled drug delivery systems, osmotic pressure-controlled drug delivery systems, etc. Such compositions are well-known to the skilled artisan and include e.g. diffusion-controlled drug delivery systems, osmotic pressure controlled drug delivery systems, erodible drug delivery systems etc. Moreover, there are pharmaceutical companies that based on a specific technology (such as mentioned above) can provide a specific composition with specific release characteristics of the active substance. Accordingly, a person skilled in the art will know how to obtain a suitable product once he has realized a specific need in respect of a particular drug substance. By way of example, Eurand is one of such companies that offer technical solutions in order to obtain a controlled release pharmaceutical composition containing a specific active substance and having specific requirements with respect to the release of the active substance from the composition (see e.g. http://wvww.eurand.com). Another company is MacroMed, Inc. that has developed a technology involving a so-called SQZgel™ (http://macromed.com, SQZgel™'s mechanism of action is a pH-sensitive polymer mixture combined with an outer coating. In the acidic environment of the stomach the polymer imbibes with water and swells, entrapping the drug. Upon entering the higher pH of the intestines, the polymer slowly shrinks, or “squeezes” at a “dialed-in” rate releasing the active composition in a sustained manner), or Egalet a/s that has a specific extrusion based technology (http://www.egalet.com, Key elements of the Egalet® technology are a biodegradable coat and a matrix, comprising the active drug, which is surface erodible, hydrophobic and composed of PEG-stearate. One of the Egalet® technologies is the 2K Egalet® constant release system, which is a 2-component production model consisting of coat and matrix. The drug is evenly distributed throughout the Egalet® matrix for constant release over time. Also of interest in the present context are technologies like e.g. the Eurand technologies Diffucaps (Drug release profiles are created by layering active drug onto a neutral core such as sugar spheres, crystals or granules followed by a rate-controlling, functional membrane. Diffucaps/Surecaps beads are small in size, approximately 1 mm or less in diameter. By incorporating beads of differing drug release profiles into hard gelatin capsules, combination release profiles can be achieved), Diffutabs (The Diffutab technology incorporates a blend of hydrophilic polymers that control drug release through diffusion and erosion of a matrix tablet), Minitabs (Eurand Minitabs are tiny (2 mm×2 mm) tablets containing gel-forming excipients that control drug release rate. Additional membranes may be added to further control release rate), Orbexa (This technology produces beads that are of controlled size and density with a defined-based granulation extrusion and spheronization techniques. The resultant beads can be coated with release rate controlling membranes for additional release rate control and may be filled into capsules or provided in sachet form) and SDS (Eurand's SDS technology uses functional polymers or a combination of functional polymers and specific additives, such as composite polymeric materials, to deliver a drug to a site of optimal absorption along the intestinal tract. In order to achieve this, Eurand first produces multiparticulate dosage forms such as Diffucaps or Eurand Minitabs, which incorporate the active drug. These dosage forms are then coated with pH dependent/independent polymeric membranes that will deliver the drug to the desired site. These are then filled into hard gelatin capsules).

Another interesting technology for use in formulating compositions according to the present invention is the so-called MeltDose® technology as described in WO 03/004001 (see http://www.lifecyclepharma.com. MeltDose® involves formulating solubilized, individual molecules into tablets. By formulating individual molecules, the primary limitation of oral absorption of drugs with low water-solubility is removed, and a superior bioavailability can be attained). By employing this technology it is possible to obtain a particulate material that is suitable for processing into various pharmaceutical dosage forms e.g. in the form of pellets or tablets. Furthermore, the technology is suitable for use as it is possible to obtain a suitable release profile of the active substance, e.g. such as those release profiles described herein. In one embodiment, pellets suitable for use may have a mean particle size larger than 2000 μm. In another embodiment, pellets suitable for use may have a mean particle size of from about 0.01 μm to about 250 μm.

Another specific suitable formulation principle for use in the present context is formulation in a lipophilic environment such as, e.g., soft gelatin capsules. A suitable example of this formulation principle is Vegicaps Soft from Scherer (a soft capsule technology based on carrageenan and starch, which despite being 100% plant-derived, still offers all the key attributes of traditional soft gelatin capsules. These include a soft and flexible dosage form that provides ease of swallowing.) (For further information see http://www.rpscherer.de/page.php?paqeID=94).

A further specific example of a suitable formulation comprises the formulation of the active substance together with Vitamin E concentrate in soft or hard gelatin capsules. This formulation, in a modified form, is the basis of the commercial cyclosporine product, Neoral®, containing, among other things, corn oil-mono-di-triglycerides, polyoxyl 40 hydrogenated castor oil NF, DL-α-tocopherol USP (part of the vitamin E family), gelatin NE, glycerol, iron oxide black, propylene glycol USP, titanium dioxide USP, carmine, and alcohol in addition to cyclosporine.

Another specific example of a suitable formulation comprises the formulation of active substance together with ethanol, tocopherolethylene glycol 1000 succinate (TPGS), corn oil and wax in soft or hard gelatin capsules. This product can be a semi-solid or solid dosage form. The release rate of this formulation is dependent on degradation due to lipases in the intestine.

A further example of a suitable formulation comprises the formulation of the active substance together with ethanol, tocopherolethylene glycol 1000 succinate (TPGS), corn oil and polyglycolized glycerides (e.g. Gelucire) in soft or hard gelatin capsules. This product can be a semi-solid or solid dosage form. The release rate of this formulation is dependent on degradation due to lipases in the intestine.

A further example of a suitable formulation is an oral pulsed dose drug delivery system. This dosage form can be perceived as a modified form of the Schering Repetab tablets. A portion of the composition of the present invention is put in the core of a tablet.

The core can for example be made by conventional wet granulation or continuous granulation such as extrusion followed by compaction of the granulate into tablets. The core is then coated using an appropriate technology, preferably by airsuspension using an enteric coating polymer such as Eudragits.

The first releasing dose is compression coated on the core or air-suspension coated either with the enteric coat or on top of the enteric coat. In a embodiment of the invention, the first releasing dose is air-suspension coated with the enteric coat. In a further embodiment of the invention, the first releasing dose is compression coated on the core, in order to avoid release of the composition according to the invention prior to the degradation of the enteric coat, such degradation typically occurring at pH values higher than those found in the gastric ventricle; i.e. the degradation of the enteric coat typically occurs after passage of the gastric ventricle.

A further example of a suitable formulation is an oral sustained drug delivery system. A portion of the composition of the present invention is put in the core of a tablet.

The core can for example be made by conventional wet granulation or continuous granulation such as extrusion followed by compaction of the granulate into tablets. The core is coated using an appropriate technology, preferably by airsuspension using ethylcellulose and a hydrophilic excipient such as hydroxylpropyl cellulose (HPC).

The first releasing dose is compression coated on the core or air-suspension coated either with the enteric coat or on top of the enteric coat. In a preferred embodiment of the invention, the first releasing dose is air-suspension coated with the enteric coat. In a further embodiment of the invention, the first releasing dose is compression coated on the core, in order to avoid release of the composition according to the invention prior to the degradation of the enteric coat, such degradation typically occurring at pH values higher than those found in the gastric ventricle; i.e, the degradation of the enteric coat typically occurs after passage of the gastric ventricle.

A further example of a suitable formulation is obtained via crystal engineering, such as e.g. described in WO 03/080034, which is hereby incorporated by reference.

Accordingly, in another embodiment the composition of the invention comprises the active substance in the form of micro-crystals with hydrophilic surfaces. Furthermore, in another embodiment of the invention, the micro-crystals are filmcoated directly, in order to achieve a sustained release formulation.

Another specific example of a suitable formulation comprises complexation of the composition according to the present invention with genuine cyclodextrins and cyclodextrin-derivatives (e.g. alkyl- and hydroxyalkyl-derivatives or sulfobutyl-derivatives). The complexation is achieved in accordance with well known methods. It is contemplated that such a complexation leads to a higher solubility and a higher dissolution rate of the composition according to the invention, compared to the composition prior to complexation. Furthermore, it is contemplated that such a complexation leads to a higher bioavailability of the composition according to the invention, compared to the composition prior to complexation.

In specific embodiments, the invention relates to a controlled release pharmaceutical composition that may be administered one, two or more times daily, such as once or twice or three times daily. Furthermore, the composition may be designed so that it releases the fumaric acid ester relatively independent on pH, i.e. the release is not dependent on pH in the gastrointestinal tract. Examples of such compositions are e.g. compositions in the form of solid dosages forms (e.g. tablets, capsules, pellets, beads etc.) that are coated with a controlled release coating. Suitable materials for controlled release coatings are e.g. cellulose and cellulose derivatives including methylcellulose, ethylcellulose and cellulose acetate, or poly(ethylene-co-vinyl acetate), poly (vinyl chloride).

The release of the fumaric acid ester typically takes place in three steps from a composition coated with a diffusion controlled membrane:

i) firstly, water (from the GI tract) diffuses into the dosage form from the surroundings,
ii) secondly, at least some of the fumaric acid ester present in the dosage form dissolves by the action of water,
iii) the dissolved fumaric acid ester diffuses out of the dosage form and into the surroundings (i.e. the GI tract)

Other examples of suitable compositions are e.g. hydrogels, i.e. monolithic systems wherein the active substance is embedded in a water-swellable network polymer. Materials suitable for use include e.g. hydrophilic vinyl and acrylic polymers, polysaccharides like alginates, and poly(ethylene oxide).

In specific embodiments, a composition according to the invention has a pH controlled release (also known as a pH dependent release) of the fumaric acid ester. Normally, the release is designed so that only a small amount, if any, of the fumaric acid ester is released in the stomach (pH up to about 3), whereas the fumaric acid ester is released in the intestines (pH shifts to about 6-7). Such a pH controlled release can be obtained by providing a composition of the invention with an enteric coating (the whole composition or, if the composition is a multiparticulate composition, the individual units) or by providing a composition that releases the fumaric acid by a pH-dependent osmotic mechanism, or by employment of suitable enzymes.

Examples of suitable substances for use as enteric coating materials include polyacrylamides, phthalate derivatives such as acid phthalates of carbohydrates, amylose acetate phthalate, cellulose acetate phthalate, other cellulose ester phthalates, cellulose ether phthalates, hydroxypropylcellulose phthalate, hydroxypropylethylcellulose phthalate, hydroxypropylmethylcellulose phthalate, methylcellulose phthalate, polyvinyl acetate phthalate, poly acrylic methacrylic acid copolymers, shellac and vinyl acetate and crotonic acid copolymers, etc.

The compositions mentioned above having a pH independent release may also be formulated to release the fumaric acid ester e.g. by providing the composition with an outer layer of an enteric coating.

Furthermore, the compositions may be formulated in such a manner that an initial delay in release of the fumaric acid ester is obtained. Such a delay may be obtained e.g. by choosing an outermost coating that in a time-controlled manner degrades (e.g. erodes) and only when this outermost coating is eroded away, the release of the fumaric acid ester starts.

### Example 1

### Example 1

### Preparation of Core Tablets

Necessary precautions were taken (protective clothing with external air supply, double gloves, arm covers, breathing mask, etc.). Non-micronized dimethyl fumarate 1200 g was placed in the basket of a fluid bed granulator. 75 g hydroxypropyl cellulose HPC-SL was dissolved by stirring in 2925 g purified water and sprayed on DMF over app. 2.5 hours until 70 g HPC was sprayed. The granules were dried over 4 minutes at 29° C. and sieved through 1.1 mm. The product temperature never exceeded 30° C.

378.2 g of the dried granules were blended with 400.6 g spray-dried lactose (FlowLac 100®), 14.6 g HPC-SL and 0.9 g Aerosil with a barrel blender at 30 rpm over 15 minutes. Finally, 5.8 g magnesium stearate was added and blended over additional 10 minutes at 30 rpm. The final blend was pressed into biconvex tablets with a diameter of 8 mm and a weight of 275 mg.

### Enteric Coating:

1 kg gastric acid-resistant coating fluid was prepared by heating 350 ml purified water to 70-80° C., adding 20 g triethyl citrate, 3 g glyceryl monostearate (Cutins GMS V), 1 g Tween 80 and stirring with the UltraTurrax for 10 minutes to achieve a homogenous mixture. 427.8 g purified water was added and the mixture was stirred with a propeller stirrer until the emulsion had reached room temperature. This emulsion was then added slowly to 210 g of a Eudragit L30 D 55 dispersion. The resulting gastric acid-resistant coating fluid was sprayed on the core tablets in a fluid bed chamber at a temperature of 30° C. over app. 2.5 hours. A drying period at 30° C. for 30 minutes and a curing period at 35° C. for additional 30 minutes followed. In one batch 780 g tablets were enteric coated.

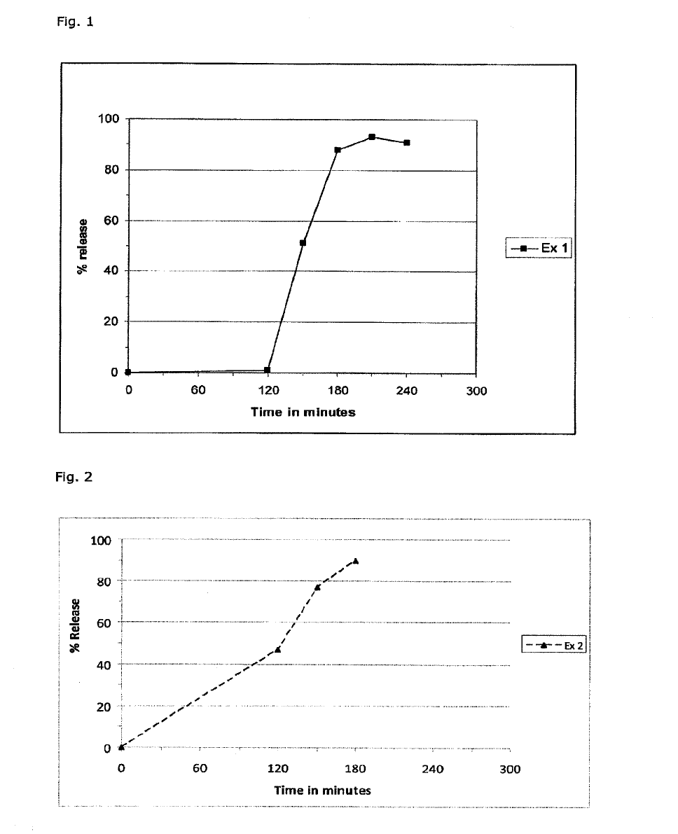

The dissolution profile of film and enteric coated tablets according to this example subjected to an in vitro dissolution test employing 0.1 N hydrochloric acid as dissolution medium during the first 2 hours of the test and then 0.05 M phosphate buffer pH 6.8 as dissolution medium appears from FIG. 1.

### Example 2

Necessary precautions were taken (protective clothing with external air supply, double gloves, arm covers, breathing mask, etc.). 1.2 kg dimethyl fumarate was sieved through a 700 μm sieve and placed in the basket of a fluid bed granulator. 70.6 g polymer hydroxypropyl cellulose HPC-SL was dissolved by stirring in 2753 g purified water and sprayed on the DMF over 2.5 to 3 hours. The granules were dried for 3 minutes at 29° C. Several batches were blended and sieved through a 700 μm sieve.

1416 g of the dried, sieved granules were blended with 1002.9 g granulated lactose (Tablettose 100 ®), 54.6 g HPC-SL and a pre-blend of Aerosil® and Tablettose® with a barrel blender at 20 rpm over 15 minutes. The pre-blend was prepared in a polyethylene bag of 3.3 g colloidal silicic acid (Aerosil®) and 501.4 g Tablettose® and sieved through 500 μm as well. Finally, 21.8 g magnesium stearate was added: The final blend was pressed into biconvex tablets with a diameter of 8 mm and a weight of 275 mg.

### Film Coating:

For film coating of 800 g core tablets a 15% suspension of Opadry was prepared by adding 36 g Opadry to 204 g purified water. App. 66% of this suspension was sprayed onto the core tablets over 35 minutes in a fluid bed chamber. The product temperature never exceeded 40° C. The coating process was followed by a drying period of 16 minutes at 30° C.

The dissolution profile of film coated tablets according to this example subjected to an in vitro dissolution test employing 0.1 N hydrochloric acid as dissolution medium during the first 2 hours of the test and then 0.05 M phosphate buffer pH 6.8 as dissolution medium appears from FIG. 2.

### Example 3

The study was a single center study, following an open-label, randomized, crossover design to investigate the plasma concentrations, pharmacokinetics, safety and tolerability of a pharmaceutical formulation according to the invention c.f. the marketed formulation Fumaderm® as reference. The tablets were administered as a single oral dose of 240 mg (2 tablets containing 120 mg each) in each treatment period according to randomization to 18 healthy, male Caucasian subjects.

Subjects were screened for eligibility at least 21 to 2 clays before first administration including: check of inclusion/exclusion criteria; demographic data (including age, body height, body weight, body mass index (BMI), and ethnic origin); physical examination; complete medical history; 12-lead electrocardiogram (ECG); vital signs (blood pressure (BP), pulse rate (PR), and body temperature (BT)); clinical laboratory parameters (hematology, serum biochemistry, and urinalysis); documentation of concomitant illness and medication.

At each treatment period, subjects came to the Study Site in the evening of Day −1 and remained there until the 24-hour blood sample for PK analysis was drawn and all safety measurements were performed (=morning of Day 2).

The subjects fasted overnight. A single oral dose (of two tablets) of the formulation according to the invention (Example 2), or two enteric-coated tablets of the reference medication Fumaderm® each containing 120 mg dimethyl fumarate (total dose 240 mg dimethyl fumarate) were administered on Day 1 (according to randomization). Administration was done to subjects who were in fasting condition together with 240 ml tap water. Between each administration, a wash-out interval of at least 7 days was maintained.

The following assessments/measurements were performed:

Blood sampling was performed for the determination of plasma concentrations and PK-parameters prior to, and at pre-scheduled times post dosing.
Adverse events were documented in detail throughout the study.
Urine was collected prior to and at pre-scheduled times post dosing.
A follow-up examination was performed at least 7 days after the last administration, including: physical examination; vital signs (BP, PR, and BT); body weight; 12-lead ECG; clinical laboratory parameters (haematology, serum biochemistry, and urinalysis); documentation of concomitant medication and adverse events.

The results of the study are shown in Table I and Table II below.

TABLE I
Coefficients of variation in % (CV).

| | Example 2 | |
|---|---|---|
| | formulation tablet | Fumaderm ® |
| AUC | 31% | 52% |
| Cₘₐₓ | 29% | 61% |

TABLE II
Summary Table: Percentage of subjects with adverse effects/side
effects after administration of the formulation according to example
2, respectively, compared to administration of Fumaderm ®

| | After administration of |
|---|---|
| | formulation acc. to ex. 2 c.f. after |
| Adverse effect/side effect | administration of Fumaderm ® |
| Flushing | 77% |
| GI related adverse effects | 65% |
| Any adverse effect | 73% |

The above results of the clinical trial show (Table II) that the tested formulation has a markedly reduced frequency of adverse effects combined with a lower variability (cf. Table I) compared to Fumaderm®.

1. A pharmaceutical composition comprising as an active substance one or more fumaric acid esters selected from di-(C₁-C₅)alkylesters of fumaric acid and mono-(C₁-C₅)alkylesters of fumaric acid, or a pharmaceutically acceptable salt thereof, wherein the release of the fumaric acid ester—when subjected to an in vitro dissolution test employing 0.1 N hydrochloric acid as dissolution medium during the first 2 hours of the test and then 0.05 M phosphate buffer pH 6.8 as dissolution medium—is as follows:
within the first 2 hours after start of the test from about 0% w/w to about 60% w/w of the fumaric ester contained in the formulation is released, and/or
within the first 3 hours after start of the test about 75% to about 95% of the total amount of the fumaric acid ester contained in the formulation is released.

2. The composition according to claim 1, wherein the release of the fumaric acid ester—when subjected to an in vitro dissolution test employing 0.1 N hydrochloric acid as dissolution medium during the first 2 hours of the test and then 0.05 M phosphate buffer pH 6.8 as dissolution medium—is as follows:
within the first 4 hours after start of the test about 92% to about 100% of the total amount of the fumaric acid ester contained in the formulation is released.

3. The composition according to any of claims 1 and 2, wherein the release of the fumaric acid ester—when subjected to an in vitro dissolution test employing 0.1 N hydrochloric acid as dissolution medium during the first 2 hours of the test and then 0.05 M phosphate buffer pH 6.8 as dissolution medium—is as follows:
within the first 5 hours after start of the test about 94% to about 100% of the total amount of the fumaric acid ester contained in the formulation is released.

4. The composition according to any of the preceding claims, wherein the release of the fumaric acid ester—when subjected to an in vitro dissolution test employing 0.1 N hydrochloric acid as dissolution medium during the first 2 hours of the test and then 0.05 M phosphate buffer pH 6.8 as dissolution medium—is as follows:
within the first 6 hours after start of the test about 95% to about 100% of the total amount of the fumaric acid ester contained in the formulation is released.

5. The composition according to any of the preceding claims, wherein the release has zero order, first order or square-root (Higuchi's) kinetics release profile.

6. The composition according to any of the preceding claims, comprising as an active substance from 30-60% by weight of one or more fumaric acid esters selected from di-(C₁-C₅)alkylesters of fumaric acid and mono-(C₁-C₅)alkylesters of fumaric acid, or a pharmaceutically acceptable salt thereof, and from 3-40% by weight of one or more rate-controlling agents.

7. The composition according to claim 6, wherein the rate-controlling agent is a cellulose polymer or a cellulose derivative or a mixture thereof.

8. The composition according to claim 6 or 7, wherein the rate-controlling agent is one or more selected from the group consisting of hydroxypropyl cellulose, hydroxypropyl methyl cellulose (HPMC), methyl cellulose, ethyl cellulose, and carboxymethyl cellulose and mixtures thereof.

9. The composition according to any of claims 6-8, wherein the rate-controlling agent is hydroxypropyl cellulose.

10. The composition according to any of the preceding claims in the form of a tablet.

11. The composition according to any of the preceding claims having one or more coatings.

12. The composition according to claim 11, wherein said coatings are film coatings and/or enteric coatings.

13. The composition according to any one of the preceding claims, wherein the fumaric acid ester is selected from the group consisting of dimethylfumarate, diethylfumarate, dipropylfumarate, dibutylfumarate, dipentylfumarate, methyl-ethyl-fumarate, methyl-propylfumarate, methyl-butylfumarate, methyl-pentylfumarate, monomethylfumarate, monoethylfumarate, monopropylfumarate, monobutylfumarate, and monopentylfumarate, including pharmaceutically acceptable salts thereof.

14. The composition according to any one of the preceding claims, wherein the fumaric acid ester is a mono-(C₁-C₅)alkylester of fumaric acid that is present in the form of a pharmaceutically acceptable salt.

15. The controlled release pharmaceutical composition according to any one of the preceding claims comprising dimethylfumarate as the active substance.

16. The controlled release pharmaceutical composition according to any one of the preceding claims comprising monomethylfumarate or a pharmaceutically acceptable salt thereof as the active substance.

17. The composition according to any one of the preceding claims for administration once, twice or three times daily.

18. The composition according to any one of the preceding claims for use in treating psoriasis, psoriatic arthritis, neurodermatitis, inflammatory bowel disease, such as Crohn's disease and ulcerative colitis, polyarthritis, multiple sclerosis (MS), juvenile-onset diabetes mellitus, Hashimoto's thyroiditis, Grave's disease, SLE (systemic lupus erythematosus), Sjögren's syndrome, Pernicious anemia, Chronic active (lupoid) hepatitis, Rheumatoid arthritis (RA), lupus nephritis, myasthenia gravis, uveitis, refractory uveitis, vernal conjunctivitis, pemphigus vuigaris, scleroderma, optic neuritis, pain such as radicular pain, pain associated with radiculopathy, neuropathic pain or sciatica/sciatic pain, organ transplantation (prevention of rejection), sarcoidosis, necrobiosis lipoidica or granuloma annuiare.

19. A method of treating psoriasis, psoriatic arthritis, neurodermatitis, inflammatory bowel disease, such as Crohn's disease and ulcerative colitis, polyarthritis, multiple sclerosis (MS), juvenile-onset diabetes mellitus, Hashimoto's thyroiditis, Grave's disease, SLE (systemic lupus erythematosus), Sjögren's syndrome, Pernicious anemia, Chronic active (lupoid) hepatitis, Rheumatoid arthritis (RA), lupus nephritis, myasthenia gravis, uveitis, refractory uveitis, vernal conjunctivitis, pemphigus vulgaris, scleroderma, optic neuritis, pain such as radicular pain, pain associated with radiculopathy, neuropathic pain or sciatica/sciatic pain, organ transplantation (prevention of rejection), sarcoidosis, necrobiosis lipoidica or granuloma annulare, which method comprises administering orally to a patient in need thereof, an effective dosage of a pharmaceutical composition according to any one of claims 1-17.

20. A use of a pharmaceutical composition according to any one of claims 1-17 for the preparation of a medicament for the treatment of psoriasis, psoriatic arthritis, neurodermatitis, inflammatory bowel disease, such as Crohn's disease and ulcerative colitis, polyarthritis, multiple sclerosis (MS), juvenile-onset diabetes mellitus, Hashimoto's thyroiditis, Grave's disease, SLE (systemic lupus erythematosus), Sjögren's syndrome, Pernicious anemia, Chronic active (lupoid) hepatitis, Rheumatoid arthritis (RA), lupus nephritis, myasthenia gravis, uveitis, refractory uveitis, vernal conjunctivitis, pemphigus vulgaris, scleroderma, optic neuritis, pain such as radicular pain, pain associated with radiculopathy, neuropathic pain or sciatica/sciatic pain, organ transplantation (prevention of rejection), sarcoidosis, necrobiosis lipoidica or granuloma annulare.
